(19) United States
(12) Patent Application Publication
(10) Pub. No.: US 2005/0249782 A1
(43) Pub. Date: Nov. 10, 2005

(54) Drug delivery device

(76) Inventors: Victor Perry, Las Vegas, NV (US); Grant Anderson, Reno, NV (US); Ly James Lee, Columbus, OH (US)

Correspondence Address: IAN F. BURNS & ASSOCIATES, P.O. BOX 71115, RENO, NV, 89570, US

(21) Appl. No.: 11/122,563

(22) Filed: May 4, 2005

Related U.S. Application Data
(60) Provisional application No. 60/568,443, filed on May 4, 2004.

(51) Int. Cl. A61K 47/00

(52) U.S. Cl. 424/439

(57) ABSTRACT

A drug delivery device having the shape and size of a pocket card and including a drug dispersed in an edible material, is disclosed. The drug delivery device may be conveniently carried by a person, such as in the form of a cash or credit card, allowing easy access to the drug. The person may administer a dosage of the drug by ingesting all or part of the drug delivery device.

### CROSS REFERENCE TO RELATED APPLICATIONS

This application claims priority of U.S. provisional patent application Ser. No. 60/568,443, filed on May 4, 2004. The above referenced application is hereby incorporated by reference in its entirety.

### BACKGROUND OF THE INVENTION

The present invention relates to a drug delivery device. In particular, the present invention provides a portable, compact drug delivery device.

As medical knowledge increases, more and more drugs are created to treat a variety of diseases, illnesses and other medical conditions. Many of these drugs need to be taken at specific times or time intervals. It may be inconvenient for people to arrange to go to where their medicines are stored at each time a dose of medicine is needed. It also may be inconvenient for people to carry standard size forms of the medicine around with them, as they can be bulky. Standard size forms of medications include designs such as tablets, capsules, caplets and pills, for example.

Some medicines need to be taken upon the occurrence of a specific situation, such as exposure to an allergen. Common situations where medicine may need to be taken spontaneously include emergency situations such as, for example, heart attacks, strokes, asthma, anxiety (panic) attacks and pain related to illness.

A blockage of blood to the heart may cause a heart attack, also known as a myocardial infarction. If the supply of blood, which carries oxygen to the heart, stops, affected portions of the heart muscle may die. This condition may cause the heart to stop pumping or for the pumping function to be impaired. Blockages may be caused by the formation of plaque, or arteriosclerosis, in blood vessels. If sections of plaque are dislodged from the walls of the blood vessels a clot may be formed.

Although the precise reasons for its effects are not fully understood, it is believed that taking aspirin (acetylsalicylic acid) at the first signs of a heart attack may reduce the severity of such a condition. The United States Food and Drug Administration and medical research have reported that use of aspirin has been indicated as reducing the risk of death from a heart attack or damage to the heart from a heart attack. However, to be most effective, aspirin must be taken at the first signs of a heart attack.

Accordingly, a need exists for a portable, convenient and secure package or delivery device for providing easily accessible dosages of drugs, such as aspirin. The conditions for which one would use such delivery devices are situations where rapid delivery of the drug is essential. For example, it is known that crushing a drug may lead to more rapid absorption of the drug by a person.

U.S. Pat. No. 6,516,950 to Robertson purports to suggest a credit card-sized carrier for a medicament. Robertson appears to provide a carrier for transporting a medicament wafer. When a dose of medication is needed, one can presumably open the carrier, remove, and ingest all or a part of the medicament wafer located inside the carrier. However, Robertson suffers from several disadvantages. First, Robertson apparently utilizes a very small container that holds a medicament wafer. Robertson contains very little discussion of how such a container might be constructed and it appears that it may be difficult and expensive to manufacture carriers of the type envisioned by Robertson. Second, the small size of the Robertson container likely would make the container difficult to open in an emergency situation. A more readily accessible form of the drug will save valuable time. Third, the small size of the container dictates that an even smaller medicament wafer must be used inside the Robertson container. It does not appear that Robertson contains any disclosure of how such thin wafers of drugs, including aspirin, may be produced. Furthermore, thin wafers of the drug may be difficult for a person to remove from the container and manipulate. The thin wafer may be brittle and break with handling. Finally, it is not clear that Robertson truly contemplates a credit card-sized drug delivery device. Robertson appears to suggest that drug delivery devices that are larger than credit cards may be used and still fit in a wallet, or otherwise be suitably transportable.

An unmet need in the art exists for a portable, easily carried drug delivery device that is easily accessed and manipulated so that a dosage of a drug may be administered as quickly as possible. It would also be beneficial for such a drug delivery device to be easily and cost effectively manufactured.

### BRIEF SUMMARY OF THE INVENTION

In one embodiment, the present invention is directed to a drug delivery device comprising an edible material about the size and shape of a pocket card, that is, a pocket-sized card. A drug is dispersed within the edible material. The drug delivery device may be conveniently carried by a person, allowing easy access to the drug. The person may administer a dosage of the drug by ingesting all or part of the drug delivery device. For example, chewing and crushing the dosage produces saliva to further enhance delivery of the drug.

The edible material of the drug delivery device may include a bottom layer having a first thickness and at least one cavity formed in the bottom layer where the cavity has a second thickness, the second thickness being less than the first thickness. The edible material of the drug delivery device may further include a top layer having a first thickness with the top layer being substantially congruent with the bottom layer. In one embodiment, at least a portion of the drug is disposed in the cavity.

In another embodiment, the present invention provides a method for delivering a drug to a person involving (a) providing a drug delivery device to be carried by a person, wherein the drug delivery device comprises an edible material substantially of the size and shape of a pocket card, and wherein the drug is dispersed within the edible material; and (b) allowing a dosage of the drug to be administered to the person by ingestion of all or a part of the drug delivery device. In a further embodiment, the present invention involves administering the dosage of the drug in response to a physical symptom corresponding to one or more emergency situations selected from the group consisting of angina, heart attack, stroke, asthma, anxiety attacks and pain related to illness. Aspirin (based on acetylsalicylic acid) may be used to alleviate heart attack symptoms, for example.

In yet another embodiment, the present invention may involve administering the dosage of the drug to the person in response to a physical symptom corresponding to one or more emergency allergic situations selected from the group consisting of insect bite (such as bee stings) hypersensitivity and food (such as peanuts) hypersensitivity. Antihistamines may by used to alleviate certain allergic reactions.

The various embodiments of the present invention may, but do not necessarily, achieve one or more of the following advantages:
the ability to quickly administer a drug;
provide a portable drug delivery device that may be carried in a purse, wallet or pocket;
provide a drug delivery device that is ingestible;
provide a drug delivery device that is palatable, that is, having a pleasing taste;
provide a drug delivery device that is sturdy and may withstand handling and manipulation;
provide a drug delivery device of which a portion may be removed and ingested in order to provide a desired drug dosage; and
provide a drug delivery device that is easily and cost-effectively manufactured.

These and other advantages may be realized by reference to the remaining portions of the specification, claims and abstract.

The above description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description or as illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1 is substantially a perspective view of one embodiment of a drug delivery device according to the present invention.

FIG. 2 is substantially a perspective view of one embodiment of a drug delivery device according to the present invention.

FIG. 3 is substantially a top view of one embodiment of a spacer for use in creating at least one embodiment of a drug delivery device according to the present invention.

### DETAILED DESCRIPTION OF THE INVENTION

In the following detailed description of certain embodiments of the present invention, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

For the purposes of the present invention, it is understood that “pocket card” or “pocket-sized card” includes cards having shapes and sizes suitable for being carried in purses, wallets or pockets, including, for example, those in the form of cash or credit cards.

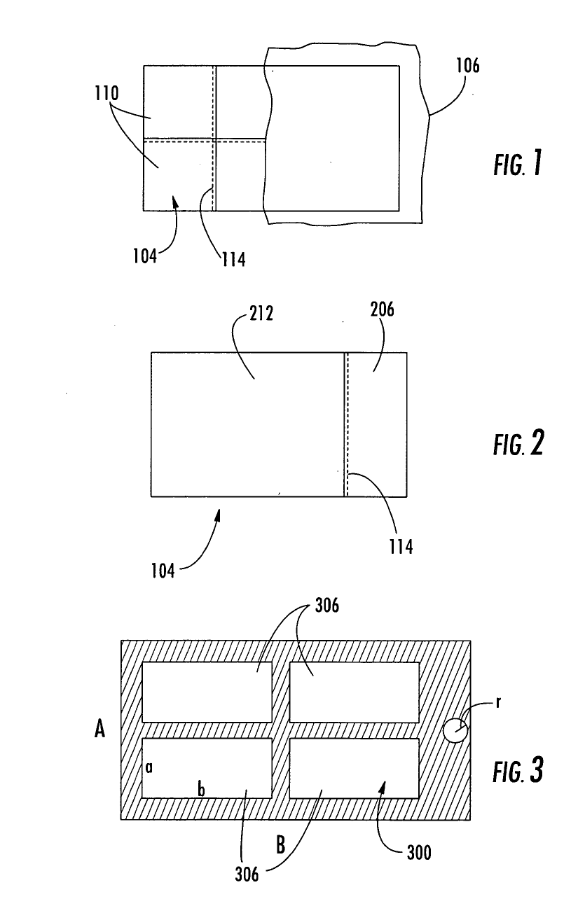

With reference to FIG. 1, the present invention is directed to a drug delivery device 104. In certain embodiments, drug delivery device 104 may be contained in a protective packaging 106, such as foil or plastic wrapping, for example. The protective packaging may serve to protect drug delivery device 104 from harmful environmental conditions such as humidity, water, heat and light; and also to draw attention to the drug delivery device via printed symbols or other information.

Drug delivery device 104 may comprise one or a plurality of dosages. If drug delivery device 104 contains multiple doses, drug delivery device 104 may be divided into a plurality of segments 110, each segment 110 may correspond to one dose or part of a dose. Segments 110 may be designed to easily be separated from the remainder of delivery device 104. For example, drug delivery device 104 may be provided with perforations 114. In the case where aspirin is the drug, a typical dosage may correspond to about 325 milligrams (mg) of aspirin. For example, if drug delivery device 104 were made up of four segments 110, then each of the segments may contain approximately 325 mg of aspirin, providing four separate doses that could be taken by a person in the time of need. Alternatively, the entire drug delivery device 104 may contain about 325 mg of aspirin, in which case the person would ingest the entire card to receive this dosage of aspirin.

Drug delivery device 104 may be of any suitable shape and dimension corresponding to that of a pocket card. A pocket card-sized drug delivery device may be advantageous as being both large enough to be sturdy and withstand handling, yet small enough to be easily transported, such as being placed in a purse, wallet or pocket. The pocket card-sized drug delivery device may be in the form of a cash or credit card; credit cards typically are dimensioned with a length of about 3.5 inches (8.9 centimeters (cm)), a width of about 2.25 inches (5.7 cm), and a thickness of about 0.05 inches (1.3 millimeters (mm)). However, pocket card-sized delivery devices of the present invention may range from about 2 to 10 cm in length, from about 1.2 to 6.5 cm in width, and from about 0.5 to 5 mm in thickness; more typically, from about 5 to 9 cm in length, from about 3 to 6 cm in width, and from about 0.7 to 2 mm in thickness;

In other embodiments, drug delivery device 104, may be smaller than a traditional credit card. For example, if each drug delivery device 104 only contains one dose of a drug, the dimensions of drug delivery device 104 may be reduced to make the drug dosage more easily ingestible. For example, drug delivery device 104 may have a similar thickness as a credit card, but be about 75% smaller in length and width, that is, about 2.2 cm by 1.4 cm in length/width, respectively. A drug delivery device 104 with these dimensions may still be easily transported, such as in a wallet or pocket, yet may constitute less material to be ingested at once. Also, this embodiment results in less wasted drug.

In other embodiments, such as shown in FIG. 2, a drug delivery device 104 may comprise a drug-containing portion 206 and an inert material 212. Drug containing portion 206 may be removably attached to inert material 212. The combined size of drug-containing portion 206 and inert material 212 may be approximately the size of a standard credit card, as described above. In this embodiment, drug delivery device 104 retains a convenient pocket-card size, but a smaller portion may be ingested to provide a drug dosage.

Drug delivery device 104 may be formed from one or more edible materials containing the desired drug dosage. The edible material may be easily ingested, broken down by the body, and may be stable for prolonged periods of time in order to give drug delivery device 104 a reasonable shelf life. Suitable edible materials include, without limitation, starches and edible, biodegradable polymers, such as polycaprolactone (PCL), polymers based on lactic acid (PLA), glycolic acid and combinations thereof (PLGA), for example. Depending on the material used for drug delivery device 104, the drug itself may be dissolved in the edible material, mixed with the edible material, or placed between layers of one or more edible materials. Suitable drug delivery devices 104 may be produced by sandwiching the desired drug, such as aspirin, between two layers of starch or edible polymer.

Although many edible materials may be used for drug delivery device 104, starch has many properties characteristic of a suitable edible material. Starch is edible and fairly inert, and therefore unlikely to adversely react with many drugs. Starch is sufficiently sturdy to withstand handling, yet is also flexible and can be made soft enough to chew. The physical and aesthetic properties of a drug delivery device 104 using starch, such as the flexibility, chewability and taste, may be altered by adding other ingredients such as cellulosics, modified starches, glycerin and sugars, for example. It is understood that other optional inactive ingredients also may be included in formulating drug delivery devices of the present invention, such as, for example, croscarmellose sodium, calcium phosphate, magnesium stearate, microcrystalline cellulose and sodium bicarbonate.

Starch is also fairly resistant to water and humidity. Water-resistant coatings may be applied to the starch-based drug delivery device 104 to further protect the drug from moisture. Although starch is not particularly light sensitive, pigment may be added to the starch to make the card opaque, and thereby help protect any light sensitive drugs that might be placed inside drug delivery device 104.

The drug may be incorporated into drug delivery device 104 in a number of ways. For example, the drug may be dispersed in the edible polymer and the card formed from the dispersion. In other embodiments, drug delivery device 104 may comprise layers of edible material that form a cavity. The drug may then be placed in the cavity. Placing the drug in the cavity may be beneficial when it is desired to have faster release of the drug. Time-release of the drug may be obtained by altering the properties of the polymer, such as by using various crosslinking agents.

### EXPERIMENTAL

The following methods provide non-limiting examples of certain embodiments of drug delivery device 104 and their preparation.

### Example I

### Starch-Based Drug Delivery Devices

A top layer was prepared by mixing 5 grams (g) of starch, 2 g of table sugar and 2 grams of glycerin with water to a total volume of 50 milliliters (ml). The mixture was heated on a hot plate at about 100° C. and stirred using manual stirring until a viscous and translucent gel formed, about 10-15 minutes. The resulting gel was poured into a 5.5 inch (14 cm) diameter dish coated with poly(dimethyl siloxane) (PDMS), which is used to help prevent the gel from sticking to the dish; fluorocarbon coatings, such as Teflon™ polymer could also be used. The gel was then dried at room temperature for 48 hours. If desired, the dish may be placed in a vacuum chamber in order remove bubbles from the gel. The size and shape of the drug delivery device 104 produced by this method may be changed and controlled by altering the size and/or shape of the dish and by altering the amount of gel used.

A base layer was prepared by mixing 10 g of starch, 5 g of table sugar, 5 g of glycerin, and adding water to give a total volume of 100 ml. The sugar and glycerin are used to adjust the brittleness of the starch and the amount used can be adjusted to achieve the desired combination of flexibility, rigidity and chewability. The mixture was heated and stirred as before until a viscous and translucent gel formed. The gel was poured into a 5.5 inch (14 cm) diameter PDMS-coated dish with two glass slide pieces at the bottom. The gel was dried at room temperature for 48 hours and a film with two cavities was obtained. The gel can then be peeled off the dish and cut to the desired dimensions.

Starch powder was used to simulate a drug. The starch powder was placed in the cavities in the base layer. The top layer was placed over the base layer and pressure was applied by placing a hard board on top of the top layer and placing a weight on top of the hard board. Although many weights could be applied, a weight of about 20 to about 40 pounds (9 to 18 kg) may be used in order secure the top layer to the base layer. The sample was then cut to approximately the size of a credit card.

### EXAMPLE II

### Poly(Lactic Acid)-Based Drug Delivery Device

Poly(lactic acid) (PLA), a biodegradable polymer, was obtained from Alkermes, Inc. in different molecular weights. In this example, PLA having a molecular weight of about 45,000 was used. It is understood that those of skill in the art will recognize that the mechanical properties of drug delivery device 104 may be altered by altering the molecular weight of the polymer used.

Because pure PLA is rather brittle, the polymer was made more pliable by modifying the polymer with a surfactant. The polymer was modified with a surfactant, Pluronic™ F127 Prill (available from BASF Corp.), by mixing the surfactant with the PLA at a ratio of 5 weight percent (wt %) surfactant in PLA in a microcompounder. The major component of the surfactant is poly(ethylene oxide)/poly(propylene oxide)/poly(ethylene oxide) (PEO-PPO-PEO) tri-block copolymer. Other surfactants, such as poly(vinyl acetate), may be used.

A mixture of PDMS resin and a curing agent (SYLGARD™ 184 kit, available from Dow Corning) in a 10:1.05 wt/wt ratio was poured into an aluminum mold spacer attached to a glass plate. Various ratios of resin to curing agent can be used, such as in the range of about 10:1.0 to about 10:1.05. FIG. 3 shows one embodiment of a suitable spacer 300 is shown in FIG. 3. The thickness of the spacer was approximately 0.6 mm. The spacer had four cavities 306; overall width and length dimensions of spacer 300 are indicated by A and B, respectively; width and length dimensions of the individual cavities are indicated by a and b, respectively; optional attachment of the card to a key-ring may be provided by a hole having a radius r. The spacer may be constructed from suitable materials such as metals and plastics. Aluminum is particularly easy to work with using conventional machining tools, such as a driller.

After the PDMS mixture was added to the mold, the mold was placed in a vacuum oven for several minutes in order to remove air bubbles formed during mixing and pouring. The PDMS mixture was then cured for two hours at 65° C. After curing, the cured PDMS mold spacer was peeled from the aluminum mold.

A PLA film with a thickness of about 1 mm was prepared via compression molding. Compression molding involves pressing a polymer melt into a mold and holding the polymer under pressure until the system cools down. The compression pressure is typically varied between 0.01 to 10 MPa (megapascals) depending on the nature the materials and the temperature. In the present method, the PLA film was placed on a pre-heated hot plate at 220° C. for 2 minutes. The PDMS mold spacer was then embossed onto the deformable PLA film and held under a pressure of about 0.5 MPa for 1 minute. The PDMS/PLA construct was then cooled to room temperature and the PDMS mold was peeled off of the PLA substrate.

Next, 0.70 g of aspirin was placed in the cavities of the PLA substrate, greater or lesser amounts of aspirin may be used. In certain embodiments, 1.0 g of aspirin may be added to the PLA substrate. A bonding solution was prepared by dissolving 1 g of PLA in 10 g of acetone to form a 10% solution by weight. The solution was used to bond the PLA substrate to a PLA cover to form a “card.” The PLA cover may be prepared by the same method as the cavities using a separate mold. The card was placed into a vacuum oven to remove the solvent.

### Example III

If desired, dyes can be added to the drug delivery device. A sample was prepared according to the procedure described in EXAMPLE II. However, 2% of acid orange food dye (obtained from Sigma-Aldrich) was added to the PLA. In this instance, 0.50 g of aspirin (total) was loaded into the sample cavities. If desired, each cavity can be made separately detachable from drug delivery device 104 by scoring or perforating the area around each cavity. Such scoring may be incorporated into a mold or may the final drug delivery device 104 may be appropriately machined.

Although the description above contains many specifications, these should not be construed as limiting the scope of the invention but as merely providing illustrations of some of the presently preferred embodiments of this invention. Thus, the scope of the invention should be determined by the appended claims and their legal equivalents rather than by the examples given.

1. A drug delivery device comprising:
(a) an edible material substantially of the size and shape of a pocket-sized card; and
(b) a drug dispersed within the edible material;
wherein the drug delivery device may be conveniently carried by a person, allowing easy access to the drug, and wherein the person may administer a dosage of the drug by ingesting all or a part of the drug delivery device.

2. The drug delivery device of claim 1 wherein the edible material comprises a bottom layer having a first thickness and at least one cavity formed in the bottom layer where the cavity has a second thickness, the second thickness being less than the first thickness.

3. The drug delivery device of claim 2 wherein at least a portion of the drug is disposed in the cavity.

4. The drug delivery device of claim 2 wherein the edible material further comprises a top layer having a first thickness, the top layer being substantially congruent with the bottom layer.

5. The drug delivery device of claim 1 wherein the drug delivery device has the dimensions of about 2 to 10 centimeters in length, about 1.2 to 6.5 centimeters in width, and about 0.5 to 5 millimeters in thickness.

6. The drug delivery device of claim 1 wherein the edible material is selected from one or more materials from the group consisting of biodegradable polymers, starch, modified starches, cellulosics, glycerin and sugars.

7. The drug delivery device of claim 6 wherein the biodegradable polymer comprises a poly(lactic acid) polymer.

8. The drug delivery device of claim 6 wherein the biodegradable polymer has been modified with a surfactant.

9. The drug delivery device of claim 1 further comprising a wrapper, wherein the wrapper completely encloses the edible material.

10. The drug deliver device of claim 9 wherein the wrapper comprises foil.

11. A method for delivering a drug to a person comprising:
(a) providing a drug delivery device to be carried by a person, wherein the drug delivery device comprises an edible material substantially of the size and shape of a pocket card, and wherein the drug is dispersed within the edible material; and
(b) allowing a dosage of the drug to be administered to the person by ingestion of all or a part of the drug delivery device.

12. The method of claim 11 wherein step (b) comprises administering the dosage of the drug to the person in response to a physical symptom corresponding to one or more emergency situations selected from the group consisting of angina, heart attack, stroke, asthma, anxiety attacks and pain related to illness.

13. The method of claim 12 wherein the drug is aspirin.

14. The method of claim 11 wherein step (b) comprises administering the dosage of the drug to the person in response to a physical symptom corresponding to one or more emergency allergic situations selected from the group consisting of insect bite hypersensitivity and food hypersensitivity.

15. The method of claim 14 wherein the drug is an antihistamine.
